(19) United States
(12) Patent Application Publication
(10) Pub. No.: US 2004/0092812 A1
(43) Pub. Date: May 13, 2004

(54) Methods and contrast agents useful in quantifying nitric oxide

(76) Inventors: Claude R. Jones, St. Louis, MO (US); James K. Bashkin, St. Louis, MO (US); Randall K. Rader, St. Charles, MO (US); John J. Kotyk, Manchester, MO (US); William L. Neumann, St. Louis, MO (US); Thomas P. Misko, St. Louis, MO (US); Christine M. Kornmeier, St. Louis, MO (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/609,023

(22) Filed: Jun. 27, 2003

Related U.S. Application Data
(60) Provisional application No. 60/392,712, filed on Jun. 28, 2002.
(60) Provisional application No. 60/392,961, filed on Jul. 1, 2002.

(51) Int. Cl. A61B 5/055

(52) U.S. Cl. 600/420; 424/9.3

(57) ABSTRACT

A contrast agent for nuclear magnetic resonance spectroscopy adapted for use in a living tissue comprising at least one reporter nucleus, together with a pharmaceutically acceptable carrier is disclosed. The contrast agent exhibits a first spectral property when not bound by nitric oxide, and a second spectral property when bound by nitric oxide. Methods of using the contrast agents for the detection of nitric oxide are also disclosed.

### CROSS REFERENCE TO RELATED APPLICATIONS

Benefit is claimed to U.S. Provisional Application serial No. 60/392,712, filed Jun. 28, 2002, and U.S. Provisional Application serial No. 60/392,961, filed Jul. 1, 2002, the disclosures of which are incorporated herein by reference.

### BACKGROUND OF THE INVENTION

Nitrogen monoxide, also called nitric oxide or NO, is an uncharged free radical that serves as a key messenger in immune, cardiovascular, and nervous systems. The physiological activity of what was later identified as NO was initially discovered in the early 1980's when it was found that vascular relaxation caused by acetylcholine is dependent on the presence of the vascular endothelium. The factor derived from the endothelium, then called endothelium-derived relaxing factor (EDRF), that mediates such vascular relaxation is now known to be NO that is generated in the vascular endothelium by one isoform of nitric oxide synthase (NOS). In addition, NO is the active species derived from known nitrovasodilators including amylnitrite, and glyceryltrinitrate. Nitric oxide is also an endogenous stimulator of soluble guanylate cyclase stimulating cGMP production. When NOS is inhibited by N-monomethylarginine (L-NMMA), cGMP formation is completely prevented. In addition to endothelium-dependent relaxation, NO is known to be involved in a number of biological actions including the cytotoxicity mediated by phagocytic cells and the strengthening of cell-to-cell communication in the central nervous system.

The identification of EDRF as NO coincided with the discovery of a biochemical pathway describing the synthesis of NO from the amino acid L-arginine by the enzyme NO synthase. There are at least three types of NO synthase as follows:

(i) a constitutive, Ca++/calmodulin dependent enzyme, located in the brain, that releases NO in response to receptor or physical stimulation;

(ii) a Ca++ independent enzyme, a 130 kD protein, which is induced after activation of vascular smooth muscle, macrophages, endothelial cells, epithelial cells, glia and a number of other cells by endotoxin and/or cytokines; and

(iii) a constitutive, Ca++/calmodulin dependent enzyme, located in the endothelium, that releases NO in response to receptor or physical stimulation.

Once expressed, inducible nitric oxide synthase (hereinafter “iNOS”) generates NO continuously for long periods. Abnormally high concentrations of NO can be very damaging and as a result play a crucial role in a variety of inflammatory responses and diseases such as osteoarthritis, rheumatoid arthritis, cancer, stroke, and coronary heart disease. Consequently, in vivo detection of nitric oxide through imaging of its distribution in human and animal tissues would provide an important biomarker for such diseases and their progression. For drug discovery and testing, quantification of nitric oxide production rates and imaging tissues like articular cartilage based on the rate at which different regions of the tissue produce nitric oxide are also needed.

Because NO is highly reactive, and therefore short lived due to its tendency to combine with superoxide radicals to form peroxynitrite, or with oxygen to nitrosylate tissue proteins, measurement of NO radicals has been limited by constraints in instrument sensitivity. Measuring nitric-oxide synthase (NOS) activity by monitoring the conversion of H³-arginine to H³-citrulline is currently the standard assay for NOS activity. NOS activity may be performed using a commercially available Citrulline Assay (NOS detect assay Kit; Stratagene, La Jolla, Calif.). Such kits typically use radiolabeled arginine, and are therefore not suitable for in vivo use.

Various complexes of iron are known to bind nitric oxide with a resulting change in the strength of the paramagnetism that they exhibit. The dithiocarbamate complexes of iron for example exhibit this behavior and are used as spin trapping agents for detection of nitric oxide by electron spin resonance (ESR) or electron paramagnetic resonance (EPR) spectroscopy and as NO activated magnetic resonance imaging (MRI) contrast agents (L J Berliner, V Khramtsov, F Hirotada, and T L Clanton, Free Radical Biol. Med. 30(5):489-499: 2001; H Fujii, X Wan, J Zhong, L J Berliner, Magn. Reson. Med 42:235-239; 1999).

The dithiocarbamate-iron-nitric oxide complexes described in the literature work fine as spin traps for EPR measurements but their use as MRI contrast agents is less promising because of the high concentrations (approaching millimolar) needed to produce useful contrast.

It is well known (S Fujii and T Yoshimura, Antioxidants and Redox Signaling, 2(4), 879-901 (2000)) that the iron-heme system in hemoglobin is a good spin trap for nitric oxide, one which can have high nitric oxide binding constants, long bound lifetimes, and ones which produce a useful EPR signal (S M Decatur, S Franzen, G D DePhillis, R B Dyer, W H Woodruff, and S G Boxer, Biochem., 35, 4939-4944 (1996)). This effect has been used for MRI research using the iron-heme system in blood (F DiSalle, P Barone, H Hacker, F Smaltion, and M d'Ischia, NeuroReport 8, 461-464 (1997)). As MRI contrast agents these compounds work by shortening the relaxation time of tissue water.

Finally, a study has been conducted to determine the location of NO binding in the heme pocket of whale myoglobin (Mb). This study employed mutant myoglobin, with two cysteines introduced at the proximal and distal surfaces of the Mb protein. The thiols of each cysteine were labeled with trifluoroacetyl group. ¹⁹F NMR of Trifluoroacetyl-Labeled Cysteine Mutants of Myoglobin: Structural Probes of Nitric Oxide Bound to the H93G Cavity Mutant M R Thomas and S G Boxer, Biochem. 40, 8588-8596 (2001). This mutant myoglobin would be unsuitable for in vivo studies, since it would be recognized by the host immune system as a foreign protein, and antibodies to the myoglobin would be expected to be raised.

### SUMMARY OF THE INVENTION

In a broad sense, the present invention relates to a method of quantifying nitric oxide using a contrast agent for nuclear magnetic resonance spectroscopy adapted for use in a living tissue having at least one reporter nucleus, together with a pharmaceutically acceptable carrier, where the contrast agent exhibits a first spectral property when not bound by nitric oxide, and a second spectral property when bound by nitric oxide. In one embodiment, the contrast agent exhibits paramagnatism when bound to nitric oxide, but does not exhibit paramagnatism when not bound to nitric oxide. In another embodiment, the contrast agent exhibits paramagnatism when not bound to nitric oxide, but does not exhibit paramagnatism when bound to nitric oxide.

In the present invention, instead of using tissue water as the source of the nuclear magnetic resonance signal for imaging, spectroscopic signals from nuclei in the complexing agent itself are used directly as the basis for analysis.

The advantage of using compounds disclosed as spectroscopic imaging agents instead of contrast agents is that the concentrations in tissue do not have to be as high. This is particularly so with the proposed fluorinated compounds. Fluorine is a very sensitivity nucleus to use in this way and has the additional advantage of the absence of an interfering fluorine background in tissues of interest. This approach also has a lot more opportunities to use synthetic chemistry to produce a large range of new and useful molecules. In addition, a characteristic of some preferred examples is optimal T1 and T2* values of the reporter nuclei in the paramagnetic state.

In a preferred example of the invention, the signals come from fluorine nuclei that are part of the chemical structure of the complexing agent. Other NMR active isotopes, such as, for example, deuterium, protons, ¹³C (carbon 13) and ³¹P (phosphorus 31), and the like, could also be used alone or in combination. The resulting signals can be used as imaging biomarkers for tissues with high rate of production of nitric oxide (utilizing MRI) and/or for spectroscopic analysis of such tissues based on chemical shift and/or relaxation in magnetic resonance spectroscopy (MRS). Most preferred examples would retain the spin trapping functionality of the previously known EPR agents. That is, the paramagnetic form of the complex induced by nitric oxide will build up to a much higher concentration than the free concentration of nitric oxide itself (this results from the paramagnetic specie having a longer lifetime in tissue than nitric oxide itself (S Pou, P Tsai, S Porasuphatana, H J Halpern, G V R Chandramouli, E D Barth, G M Rosen, Biochim. Biophys. Acta, 1427 (1999) 216-226). This gives a very large and very important boost to the sensitivity of the resulting analysis. It also has the key advantage of focusing the invention on the issue of the overall amount of nitric oxide produced in a set time period rather than the equilibrium concentration of nitric oxide.

In one embodiment of the present invention, the strength of the ligand field around the iron and the redox potential of the tissue are such that the iron is mainly in the Fe(II) oxidation state and a low spin, diamagnetic state. In this case the paramagnetism is low or non-existent. The fluorine or other reporter nuclei in the complexing agent give magnetic resonance signals which have long relaxation times and chemical shifts which differ little or not at all from their diamagnetic values. On binding nitric oxide, a strong or much stronger paramagnetic state is formed. This can have two possible effects. First the frequency of the signals from the fluorine or other reporter nuclei can be greatly changed (typically by the hyperfine shift mechanism). If the chemical shift change is large enough, this can be detected by suitably designed MRS/MRI measurements and used to generate signals specific to regions in the biological sample where the rate of nitric oxide production is high. The second useful consequence of the formation of the paramagnetic state is that the relaxation times (T1 and/or T2 and/or T1rho) of the fluorine or other reporter nuclei can be shortened. The MRS/MRI experiments can also be designed to emphasize tissues where this has occurred. In addition, shortening relaxation times can have other consequences which an MRS/MRI experiment can detect. These include the efficiency of coherence transfer (such as, for example, from ¹⁹F to ¹³C), multiple quantum coherence formation (such as, for example, ¹⁹F—¹³C multiple quantum coherence), and various consequences of cross correlation contributions to nuclear relaxation that are specific to paramagnetic systems (such as, for example, ¹⁹F—¹⁹F dipole-dipole interaction cross correlated to a ¹⁹F—paramagnetic interaction).

Another embodiment of the present invention is one in which the iron complex is paramagnetic in the absence of nitric oxide and becomes diamagnetic in its presence. This embodiment is particularly suited for use in a tissue. An example of this situation would be one in which the strength and/or the symmetry of the ligand field and the redox potential of the tissue was such that the iron was in the Fe(III) oxidation state (high or low spin) and thus paramagnetic. Reporter nuclei like fluorine show any of the consequence of paramagnetism described above. Then on binding nitric oxide the complex becomes diamagnetic and the effects described above go away. The MRS/MRI experiment can be designed and parameterized to generate images either when paramagnetism appears (above) or when it disappears due to the presence of nitric oxide in those particular regions of tissue.

Alternatively, the dinitroxide complex of Fe(III) is formed which has magnetic properties that are sufficiently unique that a MRS/MRI experiment could detect them. It is also contemplated that other metal ions besides iron could also produce such effects.

A feature of the present invention is that the molecule or complex shows a nitric oxide dependent paramagnetism which affects the spectral properties of reporter nuclei in that molecule or complexing agent and thereby produces a nuclear magnetic resonance signal from those nuclei which is useful for nuclear magnetic imaging and/or magnetic resonance spectroscopy in one of the ways exemplified above. Thus, the invention embraces any compound which, in the presence of nitric oxide, produces the desired effects thereby enabling the analysis as well as the synthetic methods which are used to prepare specific compounds.

In another embodiment of the present invention, a method of analysis of nitric oxide quantity is provided wherein a molecule capable of binding to nitric oxide and exhibiting a nitric oxide dependent paramagnetism affecting the spectral properties of at least one reporter nucleus in said molecule is contacted with a tissue or fluid, exposing the molecule to a source of nitric oxide and then measuring the paramagnetic properties of the molecule after the molecule is exposed to the nitric oxide in the tissue or fluid. Preferred means for measuring the paramagnetic properties of the molecule bound to nitric oxide include nuclear magnetic resonance and magnetic resonance imaging.

Another embodiment of the present invention is the provision of magnetic resonance measurement methods and apparatus which may be used to implement the analysis.

### DESCRIPTION OF THE INVENTION

The present invention provides improved contrast agents for the detection of nitric oxide in a sample. The contrast agent should have appropriate functionality to give paramagnetism that is nitric oxide dependent. In some preferred examples the agent contains functional groups which complex iron but at the same time leave one or more iron ligand sites open for nitric oxide to bind to. In such agents, the number, nature, and symmetry of the functional groups may be selected to modulate the oxidation potential of iron and/or the spin state for a given oxidation state and/or the binding constant of iron, and/or the transmission of hyperfine interactions to the rest of the molecule, and/or the electron relaxation time of the iron, and/or the efficiency of nitric oxide capture. Dithiocarbamates are examples of some preferred functional groups because examples of dithiocarbamates are known to hold Fe(II) mainly in its low spin diamagnetic state and to have rapid and efficient nitric oxide capture. The formation of the Fe(II)-nitric oxide pair switches this center to the paramagnetic state. Various dithiocarbamate molecules and methods of making them are known in the art, such as the compounds and methods disclosed in U.S. Pat. No. 6,407,135 issued on Jun. 18, 2002 to Lai et al., the disclosure of which is incorporated herein by reference.

The complexing agent should contain one or more reporter nuclei located close enough to the iron center in order to have their spectroscopic frequency or their relaxation times affected by the paramagnetic state of the iron/iron-nitric oxide-complex. In some preferred examples of the invention the reporter nuclei would be fluorine(s). Some such examples are in fact fluorinated analogs of known agents such as the MGD complexing agent.

Preferred complexing agents may also contain components or aspects that control the physical properties of the overall complex. An example is a functional group that enhances the overall water solubility of the complex.

Preferred complexing agents may also contain functional groups that affect the distribution of the complex in an animal in such a way that specific physical environments or specific tissues of special interest in an investigation are preferentially targeted. An example is the provision of an extra hydrocarbon chain to anchor the agent in membranes or other hydrophobic environments such as atherosclerotic plaque, for example.

Another example is the attachment of an inhibitor or drug candidate to the rest of the complexing agent. This portion of the agent then binds to an enzyme or receptor of particular interest and targets the nitric oxide analysis to tissues or regions with higher concentrations of such receptors. Similarly, an antibody to a target of interest may be attached to the rest of the complexing agent to target nitric oxide production in tissues expressing an epitope to such an antibody. Still another example is a functional group (which may or may not be well removed from the reporter nuclei and the iron binding site) which simply adds net charge to the complex. For example, extra positive charge might help target the complex to regions of articular cartilage that have high levels of the negatively charged polymer aggrecan or extra negative change to target regions of articular cartilage which are depleted in aggracan. Still further targeting functionalities are elements of bisphosphonate drugs which are known to target bone tissue. Attaching such a targeting element could help investigate the role of NO in the bone damage that can accompany arthritis. Yet another example would be the attachment of a group increasing CNS penetrability and/or localization to Alzheimer disease plaques as in the case of putresine- and beta amyloid-modified reporter molecules.

In the case of agents working on the basis of reporter nuclei relaxation changes, there are some additional characteristics that preferred examples may have. It may be preferable to avoid shortening the T2 excessively. The excitation to produce signals for MRI takes time, often 1-2 milliseconds. To avoid loss of signal, the T2* of the reporter nuclei should be longer than this time, preferably 5-20 fold longer. On the other hand the T1 values of the reporter nuclei in the paramagnetic complex should be as short as possible subject to the limitation that T2* not be shortened to the point of resulting signal loss. The short T1 values would allow for much more signal averaging of the MRI signals in the same amount of time and therefore a great increase in sensitivity. Somewhat similar considerations apply to the corresponding MRS methods except the T2* limitations are less stringent. Synthetic chemistry can be used to construct preferred examples in one of two ways. First, this could be done by placing the reporter nuclei at positions in the complexing agent where experiment shows they should have near optimal T1 and T2* values. Second, this could be done by placing multiple reporter nuclei in the general area where, a priori, near optimal T1 and T2* values would be expected with the realization that only the one or the few with near optimal values would play a constructive role in generating MRS/MRI signals.

In the example shown below, a new compound is made by replacing the N methyl group of MGD by a trifluoromethyl group. This example has the dithiocarbamate group known to complex Fe(II) in ways which are known to be productive for MRI contrast. It has the new feature of fluorines close to iron binding site so they have a good chance to feel the paramagnetic changes at that iron center. It has a chain with a number of hydroxyl groups that can help make the compound water-soluble. This particular example of the invention does not have a functional group rationally designed for targeting.

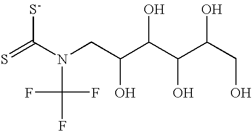

Such dithiocarbamates can form planar complexes with iron and bind nitric oxide as shown below:

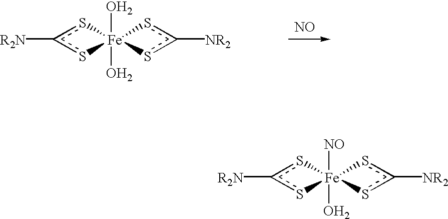

In place of the trifluoromethyl group in the structure above, various preferred examples may have longer fluorinated carbon chains. In most cases it might be best for the chain to be long enough to introduce a lot of fluorines but short enough that all of the introduced fluorines experience a strong paramagnetic effect.

The example below has extra positive charge, which could help target the complex to regions with high negative Donnan potentials (such as, for example, articular cartilage with high aggracan content). Alternatively, instead of NH₃⁺ a negative functionality like SO₃⁻ could be introduced to disfavor tissues with high negative potential. To instead target hydrophobic regions like membrane surfaces or atherosclerotic plaque, the complexing agent might have a relatively long hydrocarbon chain(s) attached to the primary amine shown below.

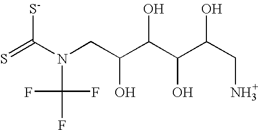

Shown below is an example in which two advantageous elements are combined into the same part of the molecule (fluorine reporter nuclei incorporated into the functionality for enhanced water-solubility). There are multiple reporter nuclei (fluorines in this case) increasing distance from the paramagnetic center but in the general area where paramagnetic effects giving optimal T1 and T2* values might be expected.

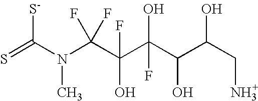

Shown below is an example with a cholesterol like targeting function to target tissues with high levels of cholesterol binding proteins as well as possible hydrophobic regions like lipoproteins and atherosclerotic plaque.

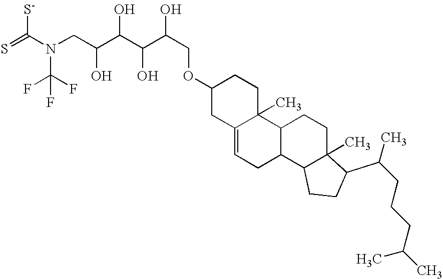

The above molecules are, of course, half of a dithiocarbamate to be complexed with a metal ion.

In another embodiment of the present invention, natural or synthetic porphyrins form a part of the contrast agent, together with at least one reporter nucleus. The porphyrin may be bound to a larger molecule, or it may be bound simply to one or more reporter nuclei.

Exemplary porphyrins include heme, and synthetic porphyrins. In another embodiment of the present invention, the heme is located in a hemoglobin molecule. In yet another embodiment of the present invention, the heme is located in a myoglobin molecule. In some circumstances it might be possible to use endogenous heme. The protons around the heme ring can show very large hyperfine shifts and could thus serve as the reporter nuclei.

Methods of making synthetic porphyrins are known in the art, such as tetramerization of monopyrroles. To synthesize porphyrins containing only one type of substituent, tetramerization of monopyrroles may be used. One approach involves the reaction between a 2,5-diunsubstituted pyrrole and an aldehyde providing the bridging methine (CH) carbons (Scheme 1). This method has also been used in the synthesis of various meso-tetraarylporphyrins, such as meso-tetraphenylporphyrin (Scheme 2).

Another approach of monopyrrole tetramerization involves the self-condensation of a 2-acetoxymethylpyrrole or 2-N,N-dimethylaminomethylpyrrole (Scheme 3). More recently, similar condensation with 2-hydroxymethylpyrroles have been carried out to synthesize various porphyrins, including porphyrins that are centrosymmetric (containing two types of substituents situated in alternate positions).

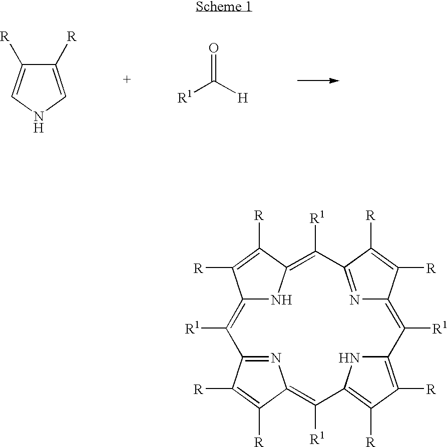

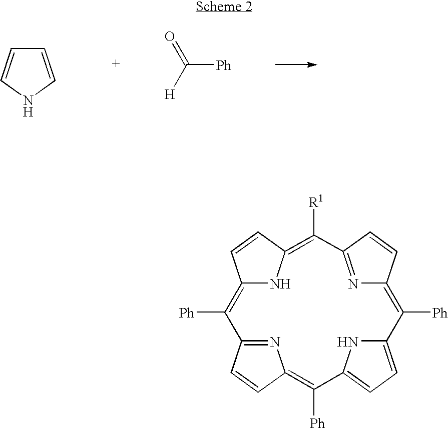

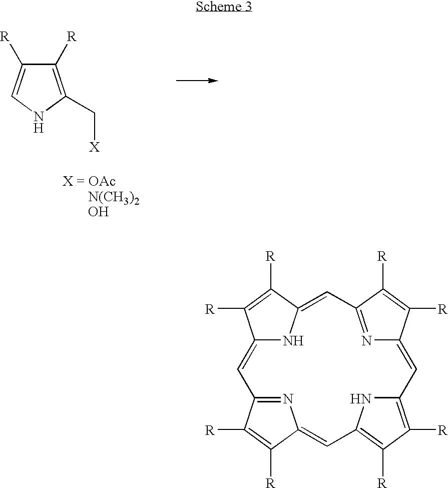

Another synthesis technique which may be advantageously employed is condensation of dipyrrolic intermediates. Developed by Fischer, the self-condensation of 1-bromo-9-methyldipyrromethenes in an organic acid melt (e.g. succinic acid) at temperatures up to 200 degrees C. gives good yields of porphyrins (Scheme 4). By condensing a 1,9-dibromodipyromethene and a 1,9-dimethyldipyrromethene, this method can also be used to synthesize porphyrins in which one or both halves of the molecule are symmetrical (Scheme 5). A variation of this method involves the reaction of 1-bromo-9-bromomethyldipyrromethenes in formic acid to give porphyrins in relatively high yields (Scheme 6).

Although known in Fischer's time, the dipyrromethane route was not widely used because of the problem of pyrrole “redistribution” during the porphyrin formation leading to a mixture of products. However, this route became common after MacDonald, in 1960, developed milder conditions for the reaction. The MacDonald synthesis involves the self condensation of 1-unsubstituted-9-formyldipyrromethanes (Scheme 7) or the condensation of a 1,9-diunsubstituted dipyrromethane and a 1,9-diformyldipyrromethane (Scheme 8) in the presence of an acid catalyst such as hydriodic acid or p-toluenesulfonic acid. This route is widely used today also because the dipyrromethanes required for the MacDonald synthesis are often more easily prepared and purified than the corresponding dipyrromethenes.

Another synthetic route involving a dipyrroketone and a dipyrromethane is less convenient than the two discussed above because the initial product obtained is an oxophlorin, which needs to be converted into a porphyrin (Scheme 9). The symmetry limitation and reaction condition follow those of the MacDonald synthesis with dipyrromethanes. It is also required that the dipyrroketone should contain the diformyl groups since 1,9-diunsubstituted dipyrroketones are not nucleophilic enough to react with 1,9-diformyldipyrromethanes.

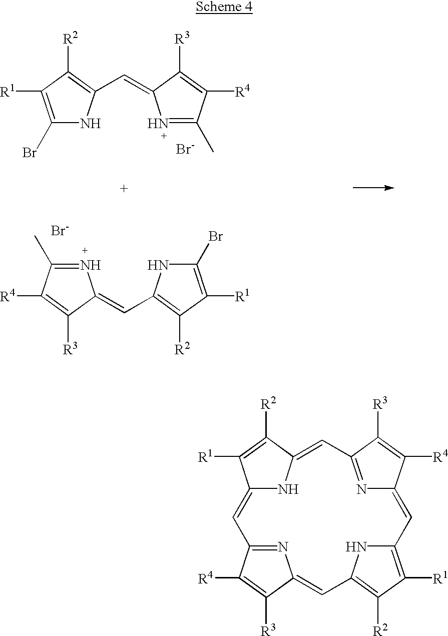

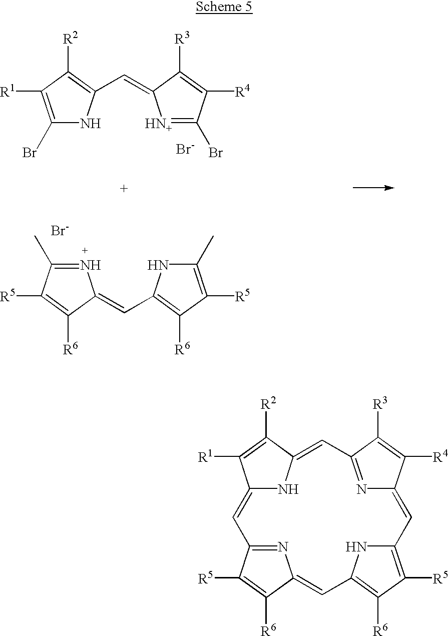

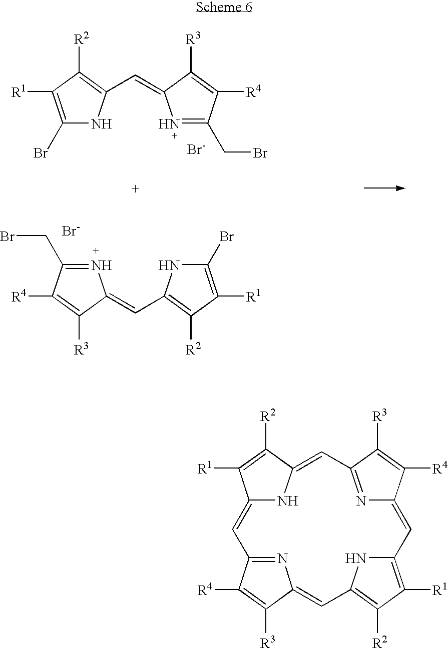

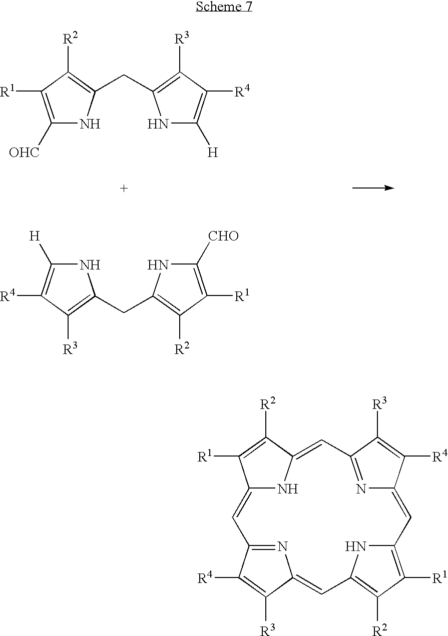

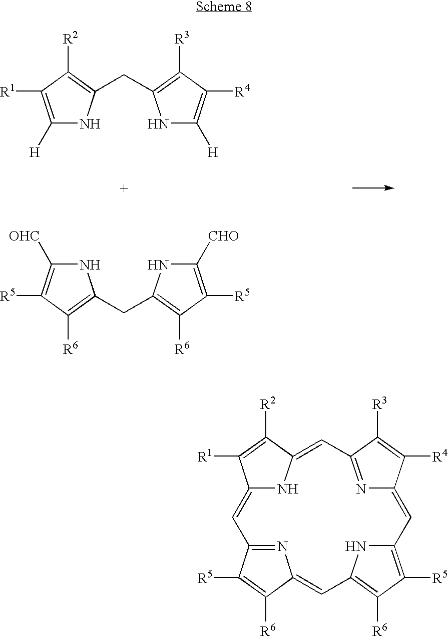

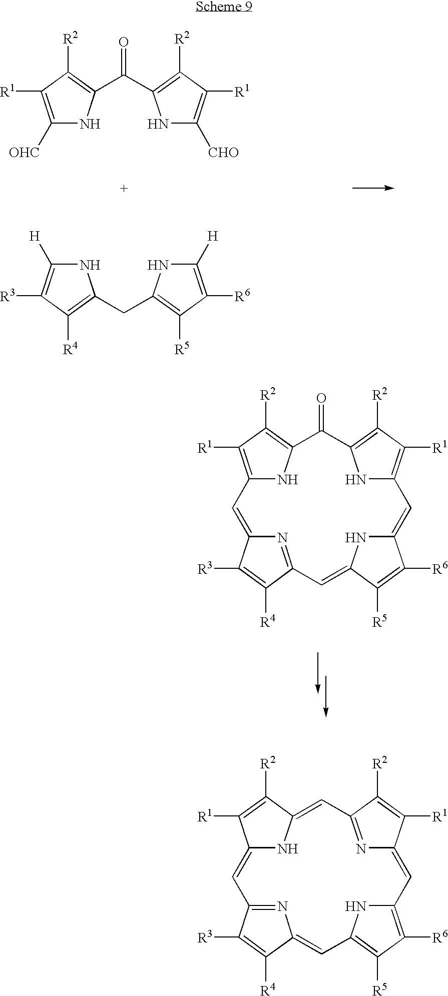

Also, porphyrins may be synthesized by cyclization of open chain tetrapyrroles, for example.

The present invention provides effective and non-invasive methods of analyzing nitric oxide quantities, and conditions mediated at least in part by pathological NOS-2 (iNOS) expression without causing untoward and unacceptable adverse effects.

Suitable subjects for the administration of the formulation of the present invention include primates, man and other animals, particularly man and domesticated animals such as cats and dogs.

For systemic use in subjects, the compounds of the invention can be formulated as pharmaceutical or veterinary compositions. Depending on the subject to be treated, and the mode of administration, the compounds are formulated in ways consonant with these parameters. The compositions of the present invention comprise a dosage effective for imaging nitric oxide. The contrast agents of this invention are preferably used in combination with a pharmaceutically acceptable carrier.

The term “pharmaceutically acceptable salt, ester, amide, and prodrug” as used herein refers to those carboxylate salts, amino acid addition salts, esters, amides, and prodrugs of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The term “salts” refers to the relatively nontoxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like.

The compositions of the present invention may be incorporated in conventional pharmaceutical formulations (e.g. injectable solutions) for use in imaging nitric oxide in humans or animals. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques.

For example, a parenteral composition may comprise a sterile isotonic saline solution containing between 0.1 percent and 90 percent weight to volume of the contrast agent.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, granules and gels. In such solid dosage forms, the contrast agent may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of contrast agent that may be combined with the carrier materials to produce a single dosage form will vary depending upon the host treated and the particular mode of administration. The selection of dosage depends upon the dosage form utilized, the condition being analyzed, and the particular purpose to be achieved according to the determination of those skilled in the art.

The dosage regimen for analyzing a disease condition with the contrast agent and/or contrast agents of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized. Thus, the dosage regimen actually employed may vary widely and therefore may deviate from the dosage regimen set forth above.

The pharmaceutical compositions of the present invention are preferably administered to a human. However, besides being useful for human nitric oxide imaging, these agents are also useful for veterinary analysis of companion animals, exotic animals and farm animals, including mammals, rodents, avians, and the like. More preferred animals include horses, dogs, cats, sheep, and pigs.

In accordance with the invention the contrast agents (or mixtures thereof) are administered in a pharmaceutically acceptable carrier in sufficient concentration so as to deliver an effective amount of the active compound or compounds to the subject tissue. Preferably, the pharmaceutical solutions contain one or more of the contrast agents in a concentration range of approximately 0.0001% to approximately 10% (weight by volume) and more preferably approximately 0.0005% to approximately 1% (weight by volume).

Any method of administering drugs directly to the subject tissue, such as to a mammalian eye may be employed to administer, in accordance with the present invention to the tissue to be treated. Suitable routes of administration include systemic, such as orally or by injection, topical, periocular (such as, for example, subTenon's), subconjunctival, intraocular, subretinal, suprachoroidal, and retrobulbar. By the term “administering directly” is meant those general systemic drug administration modes, e.g., injection directly into the patient's blood vessels, oral administration and the like, which result in the contrast agents being systemically available. More preferably, the contrast agent is injected directly into the tissue.

Various preservatives may be used in the pharmaceutical preparation. Preferred preservatives include, but are not limited to, benzalkonium chloride, chlorobutanol, thimerosal, phenylmercuric acetate, and phenylmercuric nitrate.

Likewise, various preferred vehicles may be used in topical administration, including, but are not limited to, polyvinyl alcohol, povidone, hydroxypropyl methyl cellulose, poloxamers, carboxymethyl cellulose and hydroxyethyl cellulose.

Tonicity adjustors may be added as needed or convenient. They include, but are not limited to, salts, particularly sodium chloride, potassium chloride etc., mannitol and glycerin, or any other suitable or acceptable tonicity adjustor.

Various buffers and means for adjusting pH may be used so long as the resulting preparation is pharmaceutically acceptable. Accordingly, buffers include but are not limited to, acetate buffers, titrate buffers, phosphate buffers, and borate buffers. Acids or bases may be used to adjust the pH of these formulations as needed.

In a similar vein pharmaceutically acceptable antioxidants include, but are not limited to, sodium metabisulfite, sodium thiosulfate, acetylcysteine, butylated hydroxyanisole, and butylated hydroxytoluene.

One skilled in the art will appreciate that suitable methods of administering a contrast agent which is useful in the present invention are available. Although more than one route can be used to administer a particular contrast agent, a particular route can provide a more immediate and more effective reaction than another route. Accordingly, the described routes of administration are merely exemplary and are in no way limiting.

The dose administered to an animal, particularly a human, in accordance with the present invention should be sufficient to effect the desired response in the animal over a reasonable time frame. Hence, the pharmaceutical compositions of the invention are prepared in appropriate dosage unit forms. One skilled in the art will recognize that dosage will depend upon a variety of factors, including the strength of the particular contrast agent employed, the age, species, condition or disease state, and body weight of the animal, as well as the amount of tissue expressing nitric oxide or the amount and location of nitric oxide present. The size of the dose also will be determined by the route and timing of administration as well as the existence, nature, and extent of any adverse side effects that might accompany the administration of a particular contrast agent. It will be appreciated by one of ordinary skill in the art that various conditions or disease states, in particular, chronic conditions or disease states, may require more than one quantification of nitric oxide, involving multiple administrations.

Suitable doses can be determined by conventional range-finding techniques known to those of ordinary skill in the art.

The above examples are intended to illustrate the present invention, but in no way limits the scope of the appended claims. Numerous variations will occur to those skilled in the art in light of the foregoing description.

What is claimed is:

1. A method of analysis of nitric oxide quantity comprising:
providing a molecule capable of binding to nitric oxide and exhibiting a nitric oxide dependent paramagnetism affecting the spectral properties of at least one reporter nucleus in said molecule;
contacting said molecule with a tissue or fluid, exposing said molecule to a source of nitric oxide; and
measuring the paramagnetic properties of said molecule after said molecule is exposed to said nitric oxide in said tissue or fluid.

2. The method of claim 1 wherein said molecule contains a metal atom.

3. The method of claim 2 wherein said metal atom is iron.

4. The method of claim 3 wherein said iron is in the +2 ionization state.

5. The method of claim 1 wherein said method is performed in an animal.

6. The method of claim 5 wherein said animal is a mammal.

7. The method of claim 6 wherein said mammal is a human.

8. The method of claim 1 wherein said molecule is naturally occurring.

9. The method of claim 8 wherein said molecule is synthetic.

10. The method of claim 3 wherein said molecule comprises a porphyrin together with at least one reporter nucleus.

11. The method of claim 10 wherein said porphyrin is a heme group.

12. The method of claim 11 wherein said heme group is located in a hemoglobin molecule.

13. The method of claim 11 wherein said heme group is located in a myoglobin molecule.

14. The method of claim 10 wherein said porphyrin is synthetic.

15. The method of claim 2 wherein said molecule is a dithiocarbamate, together with at least one reporter nucleus.

16. The method of claim 15 wherein said dithiocarbamate is bound to a functional group, said functional group providing one of: solubility, target tissue affinity, or tissue permeability.

17. The method of claim 1 wherein said molecule is selected from the group consisting of dithiocarbamates of the following:

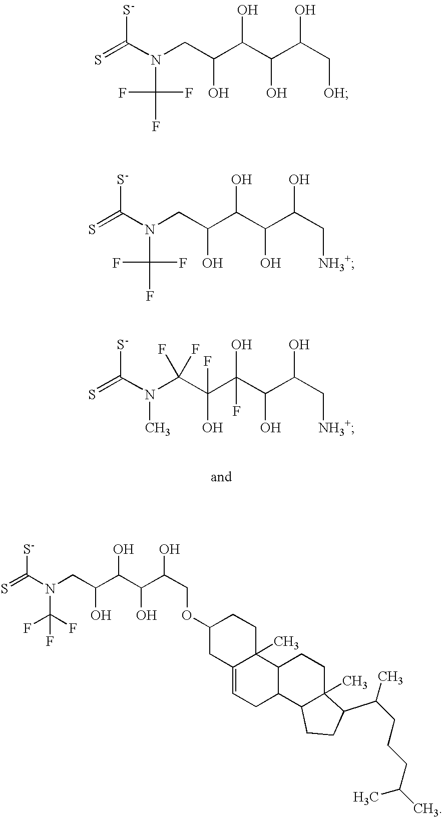

18. The method of claim 1 wherein said measurement of paramagnetic properties is performed by nuclear magnetic resonance.

19. The method of claim 1 wherein said measurement of paramagnetic properties is performed by nuclear magnetic imaging.

20. The method of claim 19 wherein said exposure to nitric oxide is in a tissue.

21. A contrast agent for nuclear magnetic resonance spectroscopy adapted for use in a living tissue comprising at least one reporter nucleus, together with a pharmaceutically acceptable carrier, said contrast agent exhibiting a first spectral property when not bound by nitric oxide, and a second spectral property when bound by nitric oxide.

22. The contrast agent of claim 21 wherein a plurality of reporter nuclei are present in said contrast agent.

23. The contrast agent of claim 21 wherein said reporter nucleus is selected from the group consisting of ¹⁹F, ¹³C, ³¹P, and deuterium.

24. The contrast agent of claim 21 wherein nitric oxide is complexed with a metal ion in said contrast agent.

25. The contrast agent of claim 24 wherein said metal ion is an iron atom.

26. The contrast agent of claim 25 wherein said iron atom is in the Fe⁺² oxidation state.

27. The contrast agent of claim 24 wherein said contrast agent comprises a porphyrin molecule.

28. The contrast agent of claim of 21 wherein said living tissue is in an animal.

29. The contrast agent of claim 28 wherein said animal is a mammal.

30. The contrast agent of claim 29 wherein said mammal is a human.

31. The contrast agent of claim 21 wherein said contrast agent comprises a dithiocarbamate together with at least one reporter nucleus.

32. The contrast agent of claim 21 wherein said dithiocarbamate contains a plurality of reporter nuclei.

33. The contrast agent of claim 21 wherein said contrast agent is selected from the group consisting of dithiocarbamates of the following:

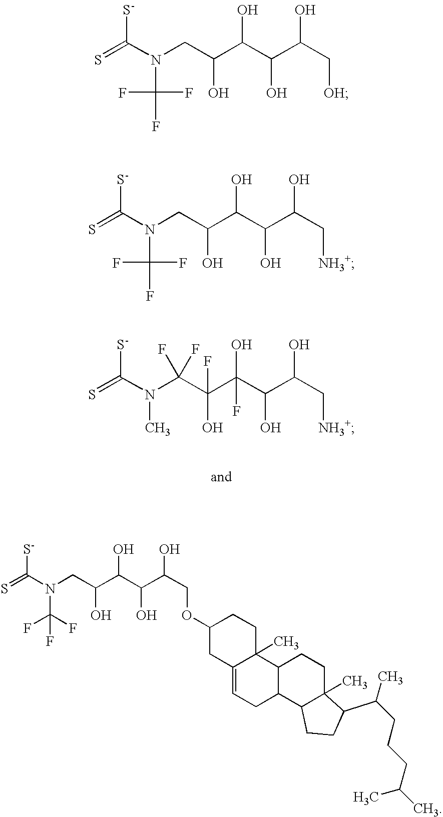

34. The contrast agent of claim 21 wherein said contrast agent comprises a porphyrin together with at least one reporter nucleus.

35. The contrast agent of claim 34 wherein said porphyrin is a heme group.

36. The contrast agent of claim 35 wherein said heme group is located in a hemoglobin molecule.

37. The contrast agent of claim 35 wherein said heme group is located in a myoglobin molecule.

38. The contrast agent of claim 34 wherein said porphyrin is a synthetic porphyrin.
